(19) United States
(12) Patent Application Publication
(10) Pub. No.: US 2010/0034869 A1
(43) Pub. Date: Feb. 11, 2010

(54) BLOCK-POLYMER MEMBRANES FOR ATTENUATION OF SCAR TISSUE

(76) Inventors: Joerg Tessmar, Regensburg (DE); Thomas Reintjes, Regensburg (DE)

Correspondence Address: STOUT, UXA, BUYAN & MULLINS LLP, 4 VENTURE, SUITE 300, IRVINE, CA, 92618, US

(21) Appl. No.: 12/480,655

(22) Filed: Jun. 8, 2009

Related U.S. Application Data
(63) Continuation-in-part of application No. 12/199,760, filed on Aug. 27, 2008.
(60) Provisional application No. 61/059,795, filed on Jun. 8, 2008.
(60) Provisional application No. 60/966,782, filed on Aug. 27, 2007.
(60) Provisional application No. 60/966,861, filed on Aug. 29, 2007.

(51) Int. Cl. A61K 9/70; A61P 41/00

(52) U.S. Cl. 424/426

(57) ABSTRACT

Precut, user-shapeable, resorbable polymer micro-membranes are disclosed. The micro-membranes are constructed of resorbable polymers, which are engineered to attenuate adhesions and to be absorbed into the body relatively slowly over time. The membranes can formed to have very thin thicknesses, for example, thicknesses between about 0.010 mm and about 0.300 mm, while maintaining adequate strength. The membranes can be extruded from polylactide polymers having a relatively high viscosity property, can be stored in sterile packages, and can be preshaped with relatively high reproducibility during implantation procedures.

### CROSS-REFERENCE TO RELATED APPLICATIONS

This application claims the benefit of U.S. Provisional Application No. 61/059,795, filed Jun. 8, 2008 and entitled Block-Polymer Membranes for Attenuation of Scar Tissue (Att. Docket MB8110PR), is a continuation-in-part of U.S. application Ser. No. 12/199,760, filed Aug. 27, 2008 and entitled Resorbable Barrier Micro-Membranes for Attenuation of Scar Tissue During Healing (Att. Docket MB8039P), and is related to U.S. application Ser. No. 10/385,399, filed Mar. 10, 2003 and entitled Resorbable Barrier Micro-Membranes for Attenuation of Scar Tissue During Healing (Att. Docket MA9496CON), now U.S. Pat. No. 6,673,362, the contents each and all of which are expressly incorporated herein by reference.

This application is also related to U.S. application Ser. No. 10/631,980, filed Jul. 31, 2003 (Att. Docket MA9604P), U.S. application Ser. No. 11/203,660, filed Aug. 12, 2005 (Att. Docket MB9828P), U.S. application Ser. No. 10/019,797, filed Jul. 26, 2002 (Att. Docket MB9962P), U.S. Provisional Application No. 60/966,782, filed Aug. 27, 2007 (Att. Docket MB8039PR), and U.S. Provisional Application No. 60/966,861, filed Aug. 29, 2007 (Att. Docket MB8039PR2). The foregoing applications are commonly assigned and the entire contents of each and all of them are expressly incorporated herein by reference.

### BACKGROUND OF THE INVENTION

1. Field of the Invention

The present invention relates generally to medical implants and, more particularly, to resorbable membranes and methods of using the membranes and of their use as medical implants.

2. Description of Related Art

A major clinical problem relating to surgical repair or inflammatory disease is adhesion which occurs during the initial phases of the healing process after surgery or disease. Adhesion is a condition which involves the formation of abnormal tissue linkages caused by the formation of fibrous scar tissue. These linkages can, for example, impair bodily function, produce infertility, obstruct the intestines and other portions of the gastrointestinal tract (bowel obstruction) and produce general discomfort, e.g. pelvic pain. The condition can in some instances be life threatening. One of the most common forms of adhesion occurs as a result of surgical interventions, although adhesion may occur as a result of other processes or events such as pelvic inflammatory disease, Khron's disease, peritonitis, mechanical injury, radiation treatment and the presence of foreign material.

Various attempts have been made to prevent adhesions, particularly postoperative adhesions. For example, the use of peritoneal lavage, heparinized solutions, procoagulants, modification of surgical techniques such as the use of microscopic or laparoscopic surgical techniques, the elimination of talc from surgical gloves, the use of smaller sutures and the use of physical barriers (membranes, gels or solutions) aiming to minimize apposition of serosal surfaces, have all been attempted. Unfortunately, limited success has been seen with these methods. Additionally, barrier materials, in various forms such as membranes and viscous intraperitoneal solutions designed to limit tissue apposition, have also met with limited success. These barrier materials can include cellulosic barriers, polytetrafluoroethylene materials, and dextran solutions.

U.S. Pat. No. 5,795,584 to Tokahura et al. discloses anti-adhesion or scar tissue reduction films or membranes, and U.S. Pat. No. 6,136,333 to Cohn et al. discloses similar structures. In the Tokahura et al. patent, a bioabsorbable polymer is copolymerized with a suitable carbonate and then formed into a non-porous single layer adhesion barrier such as a film. In the Cohn et al. patent, a polymeric hydrogel for anti-adhesion is formed without crosslinking by using urethane chemistry. Both of these patents involved relatively complex chemical formulas and/or reactions resulting in particular structures for use as surgical adhesion barriers. There continues to be a need to for an improved membrane.

### SUMMARY OF THE INVENTION

The present invention provides an improved resorbable micro-membrane that can be used in various surgical contexts, for example, to inhibit, retard, or prevent tissue adhesions and reduce scarring, e.g., during tissue healing, and then be absorbed or dissolved after an appropriate period of time. The membranes can formed to have very thin thicknesses, for example, thicknesses between about 0.010 mm and about 0.300 mm, while maintaining adequate strength.

The present invention provides an improved resorbable micro-membrane that can be readily and reliably formed and positioned on, around, or in proximity to anatomical structures comprising hard or soft tissues. The membrane can be used in various surgical contexts, for example, to retard or prevent tissue adhesions and reduce scarring. Furthermore, the co-polymers of the present invention may facilitate provision of relatively simple chemical reactions and/or formulations, and/or may facilitate provision of one or more of enhanced or more controllable mechanical strength and/or accelerated or more controllable degradation relative to other, e.g., mother, poly(esters).

In accordance with one exemplary implementation of the present invention a resorbable micro-membrane can be provided comprising a substantially uniform composition of a dualblock copolymer. The dualblock copolymer can comprise a first block that may comprise, consist essentially of, or consist of one or more polylactide and/or polyglycolide (e.g., PLA, PGA, or PLGA) and a second block that may comprise, consist essentially of, or consist of one or more a polyethylene glycol (e.g., PEG). The first block, denoted as a PLA/PGA block, may comprise a hydrophobic and biodegradable PLA/PGA block, and the second block, denoted as a PEG block, may comprise a hydrophilic PEG block.

In accordance with another feature of the present invention, a resorbable micro-membrane is provided comprising, consisting essentially of, or consisting of a substantially uniform composition of a tri block copolymer, which may comprise a first block that may comprise, consist essentially of, or consist of a polylactide and/or a polyglycolide (e.g., PLA, PGA, or PLGA), a second block that may comprise, consist essentially of, or consist of one or more polyethylene glycol (e.g., PEG), and a third block that may comprise, consist essentially of, or consist of a polylactide and/or a polyglycolide (e.g., PLA, PGA, or PLGA). The first and third blocks, each denoted as a PLA/PGA block, preferably may comprise one or more hydrophobic and biodegradable PLA/PGA blocks, and the second block, denoted as a PEG block, preferably may comprise one or more hydrophilic PEG block.

When the first and third blocks are the same or share one or more common characteristics, they may both be referred to as “A” blocks, and the second block may be referred to as a “B” block.

The first PLA/PGA block and the second PEG block together may form a PLA/PGA-PEG (i.e., A-B) copolymer, and addition of the third PLA/PGA block may altogether form a PLA/PGA-PEG-PLA/PGA (i.e., A-B-A) copolymer. These PLA/PGA-PEG (and/or PLA/PGA-PEG-PLA/PGA) copolymer membranes can be formed, for example, by extrusion at, for example, an initial, relatively high viscosity property. The initially high viscosity property may facilitate reliable formation of the membrane by, for example, attenuating the occurrence of, for example, breaking or tearing of the membrane, during the extrusion process. After processing and sterilization, the viscosity or viscosity property of the polymer(s) comprising the membrane may typically be lower. Other viscosity properties (e.g., relatively high viscosity properties) can be used according to other aspects of the invention, in order, for example, to increase the strength of the PLA/PGA-PEG (and/or PLA/PGA-PEG-PLA/PGA) copolymer material during the manufacturing process, such as an extrusion process. In modified embodiments, the initial viscosity property may not be relatively high. An extrusion manufacturing process may provide the membrane with a biased molecular orientation.

According to another feature, a membrane has a first substantially-smooth surface and a second substantially-smooth surface, is non-porous, and is about 0.01 mm to about 0.300 mm thick as measured between the first substantially-smooth surface and the second substantially-smooth surface. The membrane thus can possess a varying cross-sectional thickness. For example, the membrane can comprise at least one relatively thick portion, which can form at least a segment of an edge of the membrane. In other embodiments, the membrane may have a uniform thickness.

While the apparatus and method have or will be described for the sake of grammatical fluidity with functional explanations, it is to be expressly understood that the claims, unless indicated otherwise, are not to be construed as limited in any way by the construction of “means” or “steps” limitations, but are to be accorded the full scope of the meaning and equivalents of the definition provided by the claims under the judicial doctrine of equivalents.

Any feature or combination of features described herein are included within the scope of the present invention provided that the features included in any such combination are not mutually inconsistent as will be apparent from the context, this specification, and the knowledge of one of ordinary skill in the art. In addition, any feature or combination of features described herein may be specifically excluded from any embodiment of the present invention. For purposes of summarizing the present invention, certain aspects, advantages and novel features of the present invention are described. Of course, it is to be understood that not necessarily all such aspects, advantages or features will be embodied in any particular implementation of the present invention. Additional advantages and aspects of the present invention are apparent in the following detailed description and claims that follow.

### BRIEF DESCRIPTION OF THE DRAWINGS

FIGS. 1-6 elucidate compositions and characteristics of exemplary embodiments in accordance with the present invention.

### DETAILED DESCRIPTION OF THE INVENTION

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of this disclosure, while discussing exemplary embodiments, is that the following detailed description be construed to cover all modifications, alternatives, and equivalents of the embodiments as may fall within the spirit and scope of the invention as defined by the appended claims.

Barrier membranes of the present invention may be constructed from various biodegradable materials, such as resorbable polymers. In accordance with one embodiment, non-limiting polymers which may be used to form barrier membranes of the present invention can include a dualblock copolymer. As embodied herein, the dualblock copolymer can comprise a first block that may include, consist essentially of, or consist of a polylactide and/or a polyglycolide (e.g., PLA, PGA, or PLGA) and a second block that may include, consist essentially of, or consist of a polyethylene glycol (e.g., PEG). The first block, denoted as a PLA/PGA block, can comprise one or more of a hydrophobic and a biodegradable PLA/PGA block, and the second block, denoted as a PEG block, can comprises a hydrophilic PEG block. The first PLA/PGA block may be referred to as an “A” block, and the second PEG block may be referred to as a “B” block. The first PLA/PGA block and the second PEG block together may form a PLA/PGA-PEG (i.e., A-B, or AB) dualblock copolymer.

Other non-limiting block polymers which may be used to form barrier membranes of the present invention include a triblock copolymer or a starblock copolymer. As embodied herein, the triblock copolymer can comprise a first block that may include or consist of a polylactide and/or a polyglycolide (e.g., PLA, PGA, or PLGA), a second block that may include or consist of a polyethylene glycol (e.g., PEG), and third block that may include or consist of a polylactide and/or a polyglycolide (e.g., PLA, PGA, or PLGA). The first block, denoted as a PLA/PGA block, can comprise a hydrophobic and biodegradable PLA/PGA block, the second block, denoted as a PEG block, can comprise a hydrophilic PEG block, and the third block, denoted as a PLA/PGA block, can comprise a hydrophobic and biodegradable PLA/PGA block. When the first PLA/PGA block and third PLA/PGA block are the same or share one or more common characteristics, they may each be referred to as an “A” block, and the second PEG block may be referred to as a “B” block. The first PLA/PGA block, the second PEG block, and the third first PLA/PGA block together may form a PLA/PGA-PEG-PLA/PGA (i.e., A-B-A, or ABA) triblock copolymer.

The combination block copolymer may, alternatively, be characterized as a PEG-PLA/PGA-PEG (i.e., B-A-B, or BAB) triblock copolymer.

In other implementations, the combination block copolymer may be a 4plus (i.e., four or more blocks) block copolymer, comprising, for example, a PEG block (i.e., B block) that is formed with, coupled to, or disposed between three or more PLA/PGA blocks (i.e., A blocks). The 4plus block copolymer may, alternatively, comprise, for example, a PLA/PGA block (i.e., A block) that is formed with, coupled to, or disposed between three or more PEG blocks (i.e., B blocks).

The 4plus block copolymer may comprise a PEG block (i.e., B block) having one or more of a symmetrical shape and a star shape, with regions (e.g., arms, branches, or points) coupling (e.g., being connected to or with) three or more PLA/PGA blocks (i.e., A blocks), which number may comprise, for example, four. In a preferred implementation, the number of regions equals the number of PLA/PGA blocks. Alternatively, the 4plus block copolymer can comprise a PLA/PGA block (i.e., A block) having one or more of a symmetrical shape and a star shape, with regions (e.g., arms, branches, or points) coupling (e.g., being connected to or with) three or more PEG blocks (i.e., B blocks). The number of regions can, as in the preceding example, equal the number of PEG blocks and, in a particular example, can comprise four.

The combination block copolymer membranes can be formed by extrusion at an initial, relatively high viscosity property. The initially high viscosity property may facilitate reliable formation of the membrane by attenuating the occurrence of, for example, breaking or tearing of the membrane, during the extrusion process. After processing and sterilization, the viscosity property of the membrane will typically be lower. Other relatively high viscosity properties can be used according to other aspects of the invention, in order, for example, to increase the strength of the material. The extrusion procedures advantageously can provide for efficient production of the membranes. Moreover, membranes which are manufactured by such extrusion techniques can be free from solvent trappings in the membrane and, furthermore, can be provided with, for example, a molecular bias, including a predetermined molecular bias. Monoaxial or biaxial extrusion may be employed to manufacture the membranes.

Compositions of the combination block copolymer can be extruded to form membranes of the present invention. In certain embodiments, PLA/PGA-PEG block copolymers taking the forms of one or more of the following polymers: 1. Poly(L-lactide-co-PEG), 2. Poly(L-lactide-co-DL-lactide-co-PEG), and 3. Poly(L-lactide-co-glycolide-co-PEG); PLA/PGA-PEG-PLA/PGA block copolymers taking the forms of: 4. Poly(L-lactide-co-PEG-co-L-lactide), 5. Poly(L-lactide-co-PEG-co-L-lactide-co-DL-lactide), 6. Poly(L-lactide-co-PEG-co-L-lactide-co-glycolide), 7. Poly(L-lactide-co-DL-lactide-co-PEG-co-L-lactide-co-DL-lactide), 8. Poly(L-lactide-co-DL-lactide-co-PEG-co-L-lactide-co-glycolide), 9. Poly(L-lactide-co-glycolide-co-PEG-co-L-lactide-co-glycolide), and 10. other forms from combinations and/or permutations of the above (optionally combined with any one or more other items disclosed or referenced herein) for starblock and/or 4plus block copolymers, can be manufactured or obtained. For instance, such items may be manufactured or obtained, without limitation, from Boehringer Ingelheim KG of Germany, for extrusion into the membranes of the present invention.

Exemplary chemical structures, and synthesis and nomenclature conventions to be used herein follow, wherein:

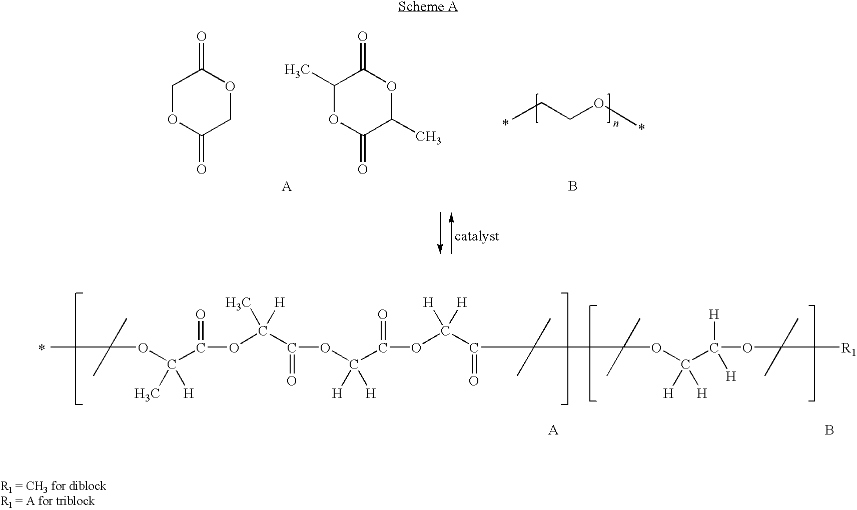

Scheme B shows the incorporation of polyethylene glycol (PEG) units into a block co-polymer with PLGA, again by the action of the catalyst. PEG also has low systemic toxicity, and is currently used in various medical and pharmaceutical agents.

The resulting block co-polymer can be represented schematically as follows:

### Scheme B

RLLRLLLRLLRLLRR—O—[—CH₂—CH₂—O—]ₙ—R

### A B

Commercially obtained PLGA:PEG block co-polymers include the RESOMER® PEG products from Boehringer Ingelheim.

One preferred (though non-exclusive) product is RESOMER® PEG Sample MD Type LRP d 70 5 5, wherein LR stands for RESOMER Acronym LR (A-block), P stands for PEG (B-block), 70 stands for the mole ratio within the A-block, the first 5 stands for the weight percent PEG, and the second 5 stands for the molecular weight of the PEG divided by one thousand.

Typical non-limiting examples of PLA/PGA-PEG (and/or PLA/PGA-PEG-PLA/PGA) copolymers are as follows: For controlled release functionalities (CR), the polymer will typically contain from about 5% to about 15% PEG. For medical devices (MD) the polymer will typically contain less than about 5% PEG. For controlled release the A block may contain, e.g., D,L-lactide-co-glycolide (RG). For Medical Devices, the A Block may contain, e.g., L lactide (L), L-lactide-co-D,L-lactide (LR), or L Lactide-co-glycolide (LG).

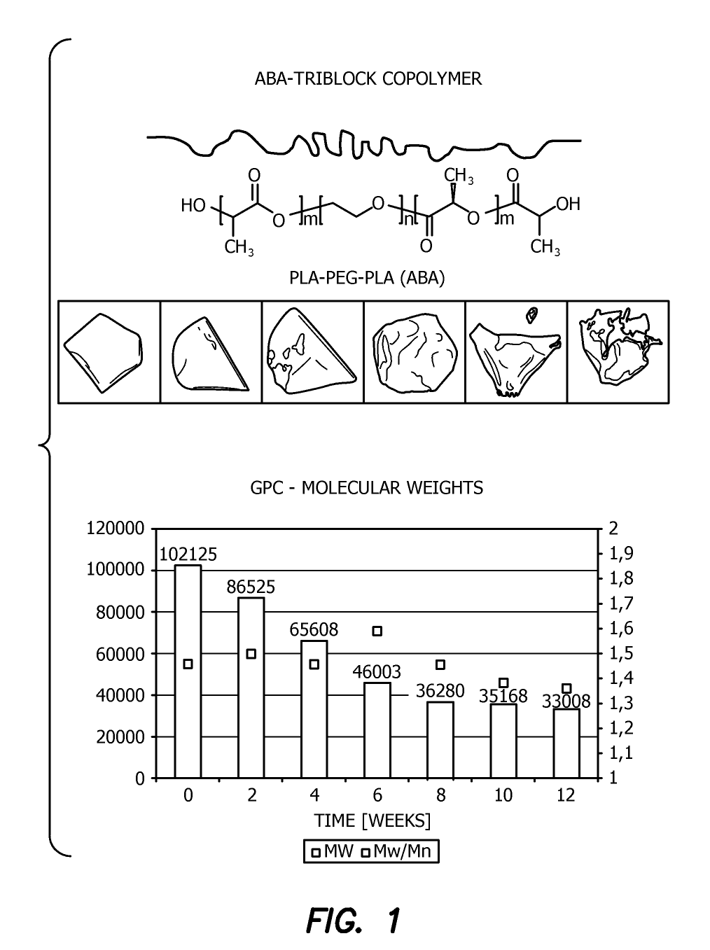

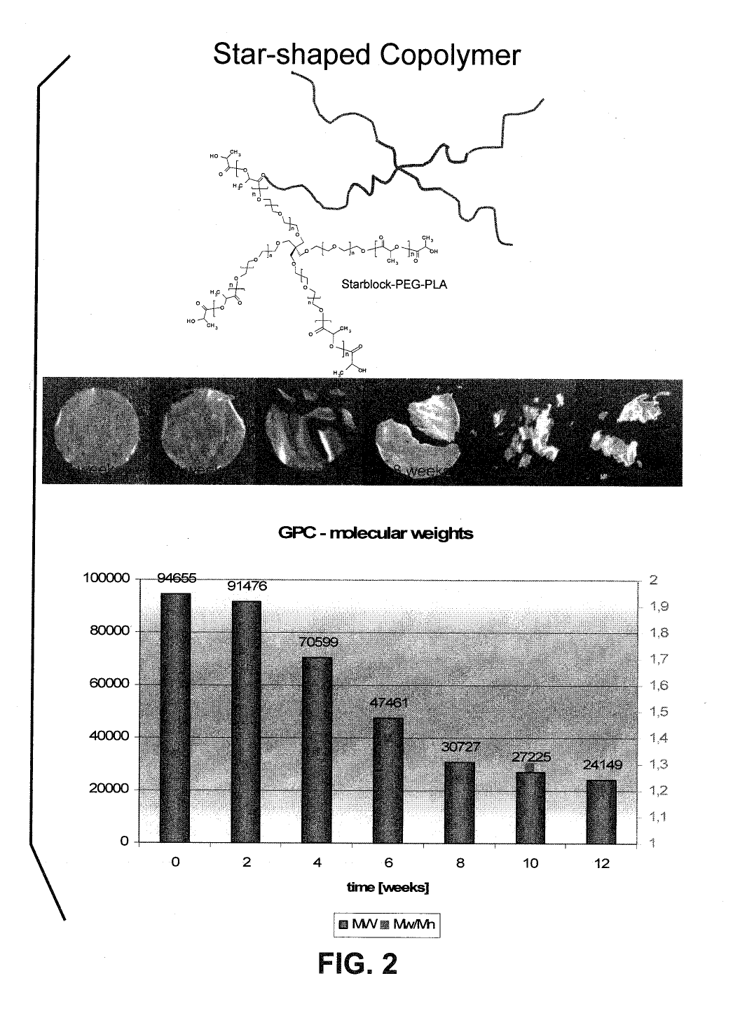

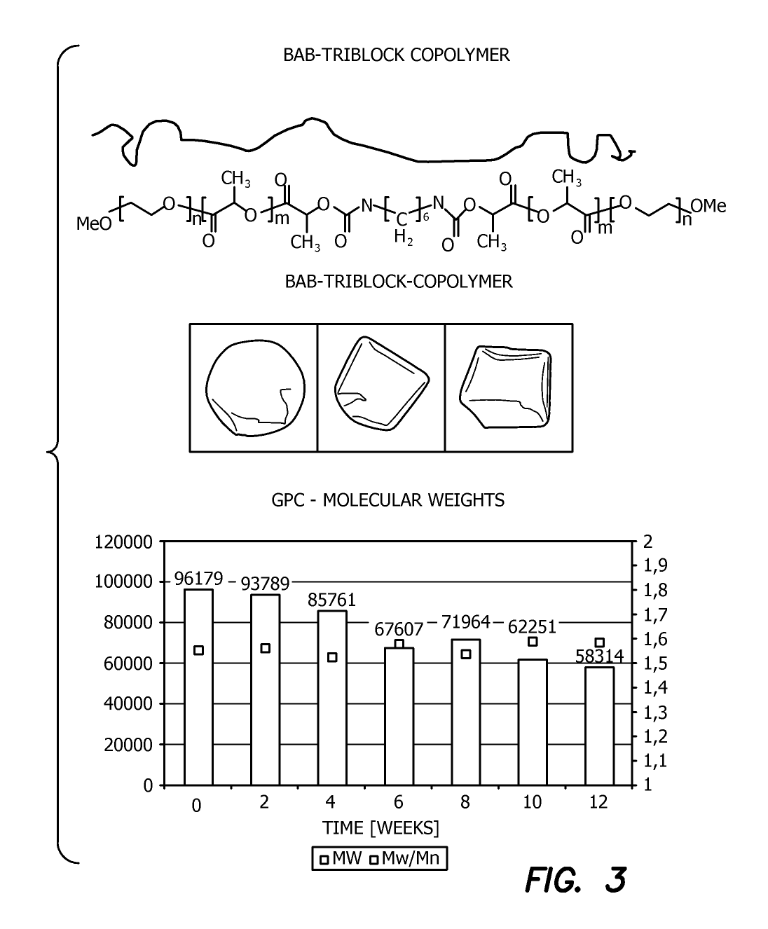

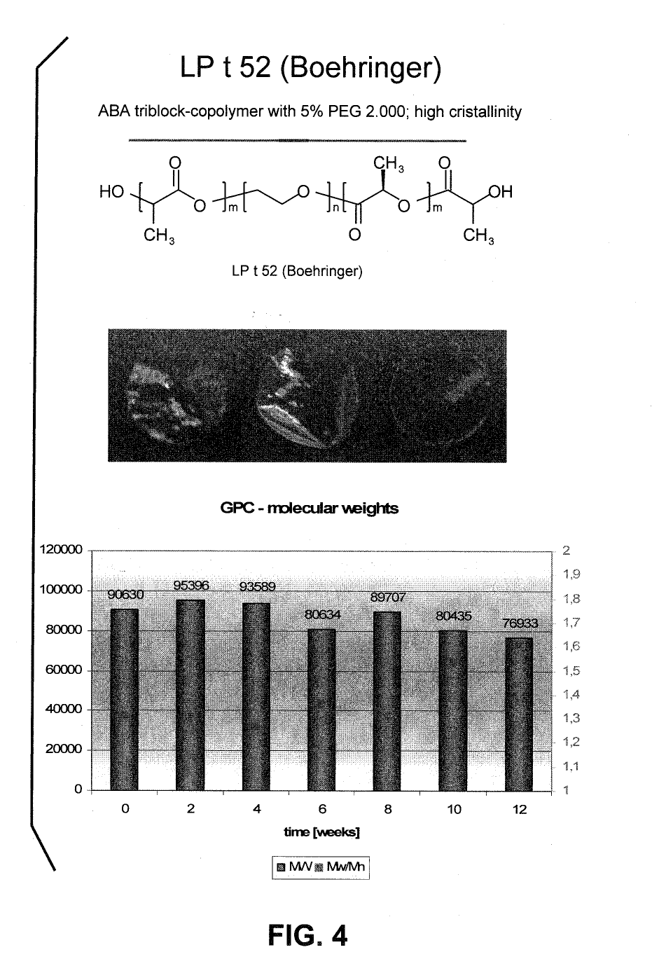

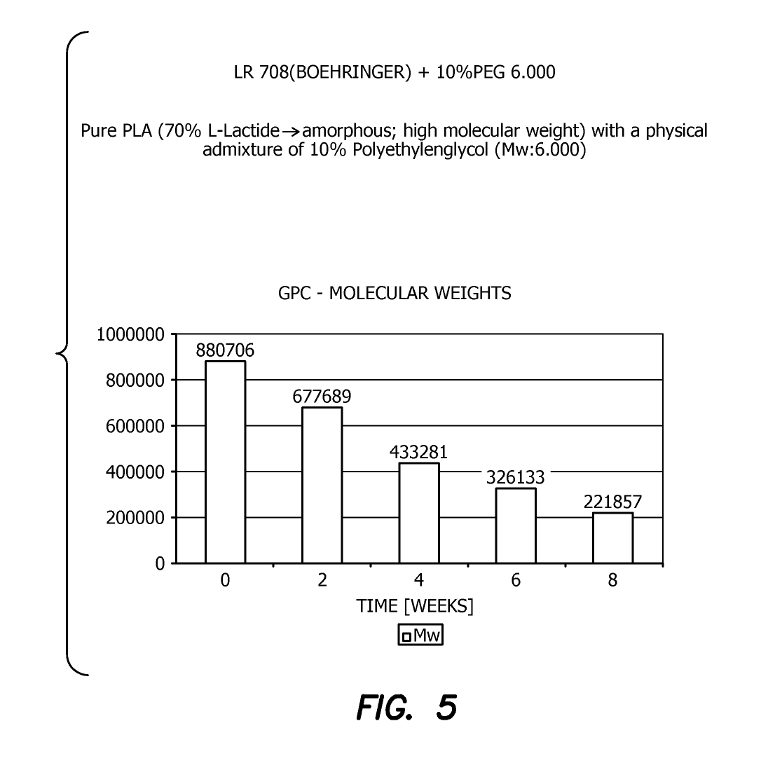

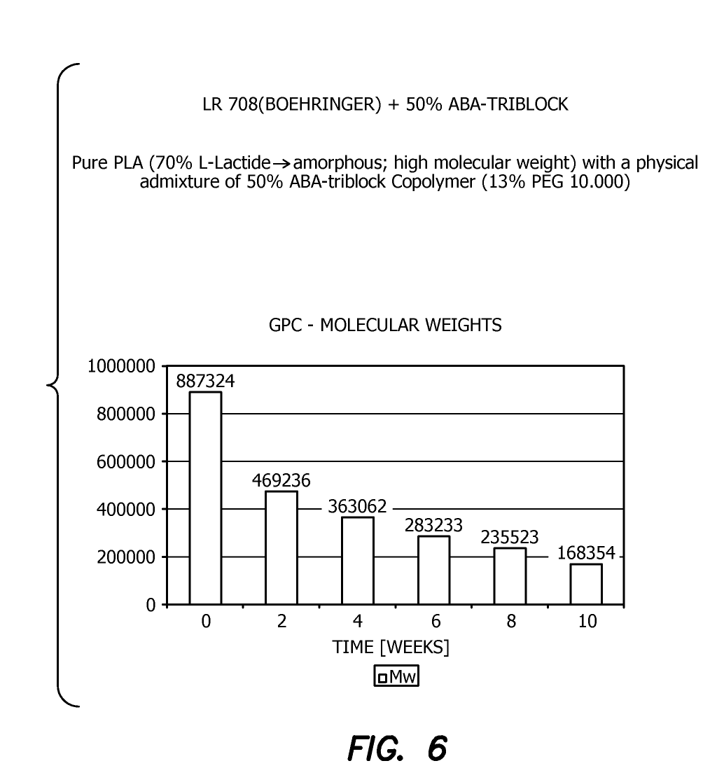

FIGS. 1-6 elucidate certain compositions and characteristics of contemplated embodiments according to the present invention. A membrane of the present invention can have at least one substantially smooth-surface. Preferably, a membrane of the present invention has two (opposing) substantially smooth surfaces. As measured between the opposing surfaces, a membrane of the present membrane can have a thickness of about 0.01 mm to about 0.3 mm and, more preferably, about 0.01 mm to about 0.1 mm. In a preferred embodiment, a membrane of the present invention has a thickness of about 0.015 mm to about 0.025 mm. In another preferred embodiment, a membrane of the present invention has a maximum thickness of about 0.02 mm. A preferred micro-membrane of the present invention can comprise one or more of a substantially uniform composition and a biased molecular orientation in the membrane as a consequence, for example, of extrusion.

As used herein, the term “non-porous” refers to a material which is generally water tight and, in accordance with a preferred embodiment, not fluid permeable. However, in a modified embodiment of the invention micro-pores (which are fluid permeable but not cell permeable) may exist in the micro-membrane of the present invention, to the extent, for example, that they do not substantially disrupt the smoothness of the surfaces of the resorbable micro-membrane to cause scarring of tissue. In substantially modified embodiments for certain applications, pores which are cell permeable but not vessel permeable may be manufactured and used.

As presently embodied, many of the thinner membrane thicknesses can be sufficiently contoured even in the absence of heating to glass transition temperature. As presently embodied, the resorption of the resorbable membrane can be between approximately 2 and 24 months. In one embodiment, membranes of the present invention can be capable of resorbing (i.e., being absorbed by the mammalian body) within a period, for example, of about 10 to 20 weeks, or of about 20 to 30 weeks, or, according to other implementations, up to about 18 months, or up to about 24 months or more from an initial implantation of the membrane into the mammalian body. The resorbable membrane can be resorbed within the body of the patient to a point where substantial strength is no longer present within a period of approximately 1 year. Complete resorption of the resorbable membrane may subsequently occur after a total period of 1.5 to 2 years has elapsed since the initial implantation. In other embodiments, the resorbable membrane may comprise in whole or part non-resorbable plastic or metallic materials.

The micro-membranes may be used in a number of surgical applications, including: surgical repair of fracture orbital floors, surgical repair of the nasal septum and perforated ear drum micro-membrane, as a protective sheathing to facilitate osteogenesis, surgical repair of the urethral anatomy and repair of urethral strictures, prevention of synostosis in completed corrective surgery for cranial fusions and forearm fractures, lessening of soft-tissue fibrosis or bony growth, as a temporary covering for prenatal rupture omphalocele during staged repair procedures, guided tissue regeneration between the teeth and gingival margin, tympanic membrane repairs, dural coverings and neural repair, heart vessel repair, hernia repair, tendon anastomoses, temporary joint spacers, wound dressings, scar coverings, and as a covering for gastroschisis. The micro-membrane of the present invention can be particularly suitable for preventing tissue from abnormally fibrotically joining together following surgery, which can lead to abnormal scarring and/or interfere with normal physiological functioning. In some cases, such scarring can force and/or interfere with follow-up, corrective, or other surgical operations.

The very thin construction of these membranes is believed to substantially accelerate the rate of absorption of the membranes, compared to rates of absorption of thicker membrane implants of the same material. It is believed, however, that resorption into the body too quickly of the membrane may, in some instances, yield undesirable drops in local pH levels, thus introducing/elevating, for example, local inflammation, discomfort and/or foreign antibody responses. Further, a resulting uneven (e.g., cracked, broken, roughened or flaked) surface of a micro-membrane degrading too early may undesirably cause tissue turbulence between the tissues before, for example, adequate healing has occurred, potentially resulting in tissue inflammation and/or scarring, as well as risking the formation of tissue adhesions, thus defeating a purpose of the membrane. In other instances, a different (e.g., more rapid) resorption may be desired in one or more areas of a patient, and/or at one or more points in time of one or more surgical procedures, so that, in accordance with an aspect of the present invention, rates of absorption may be varied, temporally and/or spatially, or contour varied, by varying the materials of the membrane or parts thereof.

Micro-membranes in accordance with an aspect of the present invention may be provided in rectangular shapes that are for example several centimeters on each side, or can be cut and formed into other specific shapes, configurations and sizes, by the manufacturer before packaging and sterilization. In modified embodiments, various known formulations and copolymers of, for example, polylactides may affect the physical properties of the micro-membrane. The micro-membranes of the present invention may be sufficiently flexible to conform over and/or around anatomical structures, although some heating in a hot water bath may be necessary for thicker configurations. In modified embodiments, certain polylactides which may become somewhat more rigid and brittle at thicknesses above, for example, 0.25 mm and which may be softened by formation with other polymers, copolymers and/or other monomers, e.g., epsilon-caprolactone, for example, may be implemented to form micro-membranes.

Moreover, in accordance with another aspect of the present invention, the micro-membrane may comprise a substance for cellular control, such as at least one of a chemotactic substance for influencing cell-migration, an inhibitory substance for influencing cell-migration, a mitogenic growth factor for influencing cell proliferation and a growth factor for influencing cell differentiation. Such substances may be disposed on and/or impregnated within the membrane, but may also be coated on one or more surfaces of the membrane. In addition, substances may be contained in discrete units on or in the membrane, which may be effective to facilitate selective release of the substances when the membrane is inserted into a patient. Other configurations for accommodating different anatomical structures may be formed. For example, configurations may be designed to be formed into, for example, cone structures to fit around base portions with protrusions extending through the centers of the membranes. Suture perforations may be formed around perimeters of the membranes, and cell and vessel permeable pores may be included as well.

In general, any particulars, features or combinations thereof (in whole or in part, in structure or step), described or referenced herein, may be combined with any particulars, features or combinations thereof (in whole or in part, in structure or step), described or referenced in any of the documents mentioned herein, including without limitation U.S. application Ser. No. 11/203,660 and U.S. Provisional Application No. 60/966,861 and/or, U.S. application Ser. No. 10/019,797 (in whole or in part, in any combination or permutation that would be viewed by one skilled in the art to be possible or modifiable to be possible, in structure or step, provided that the particulars or features included in any such combination are not mutually inconsistent. Each of these patent applications is expressly incorporated by reference herein.

In accordance with one implementation of the present invention, the pre-formed micro-membranes can be preformed and sealed in sterilized packages for subsequent use by the surgeon. Since one objective of the micro-membranes of the present invention can be to reduce sharp edges and surfaces, preformation of the membranes is believed to help, in some instances, facilitate, albeit to a relatively small degree, rounding of the edges for less rubbing, tissue turbulence and inflammation. That is, the surfaces and any sharp edges of the micro-membranes are believed to be capable of ever so slightly potentially degrading over time in response to exposure of the membranes to moisture in the air, to thereby form rounder edges. This is believed to be an extremely minor effect. Moreover, any initial heating to glass temperature of the pre-cut membranes just before implanting may conceivably further round any sharp edges. Furthermore, the very micro-membranes of the present invention may be particularly susceptible, at least theoretically, to these phenomena, and, perhaps to a more noticeable extent, are susceptible to tearing or damage from handling, thus rendering the pre-forming of the micro-membranes potentially beneficial for preserving the integrity thereof.

In accordance with an aspect of the present invention, a surgical prosthesis (e.g., a resorbable scar-tissue reduction micro-membrane system) can comprise an adhesion-resistant region (e.g., a biodegradable region, a biodegradable side, a membrane and/or a micro-membrane) as described herein, and further may comprise an optional tissue-ingrowth region (e.g., another membrane, a bridging membrane as referenced herein, a biodegradable region and/or a biodegradable side or mesh).

The surgical prosthesis (e.g., biodegradable surgical prosthesis) can be constructed for use in the repair of soft tissue defects, such as soft tissue defects resulting from incisional and other hernias and soft tissue defects resulting from extirpative tumor surgery. The surgical prosthesis may also be used in cancer surgeries, such as surgeries involving sarcoma of the extremities where saving a limb is a goal. Other applications of the surgical prosthesis of the present invention may include laparoscopic or standard hernia repair in the groin area, umbilical hernia repair, paracolostomy hernia repair, femora hernia repair, lumbar hernia repair, and the repair of other abdominal wall defects, thoracic wall defects and diaphragmatic hernias and defects.

According to an aspect of the present invention, the tissue-ingrowth region and the adhesion-resistant region may differ in both (A) surface appearance and (B) surface function. For example, the tissue-ingrowth region can be constructed with at least one of a surface topography (appearance) and a surface composition (function), either of which may facilitate strength, longevity or lack thereof, and/or a substantial fibroblastic reaction in the host tissue relative to for example the anti-adhesion region. On the other hand, the adhesion-resistant region can be constructed with at least one of a surface topography and a surface composition, either of which may facilitate, relative to the tissue-ingrowth region, an anti-adhesive effect between the biodegradable surgical implant and host tissues.

### A. Surface Topography (Appearance):

The tissue-ingrowth region can be formed to have an open, non-smooth and/or featured surface comprising, for example, alveoli and/or pores distributed regularly or irregularly. In further embodiments, the tissue-ingrowth region can be formed to have, additionally or alternatively, an uneven (e.g., cracked, broken, roughened or flaked) surface which, as with the above-described surfaces, may cause tissue turbulence (e.g., potential tissue inflammation and/or scarring) between host tissues and the tissue-ingrowth region.

Over time, with respect to the tissue-ingrowth region, the patient's fibrous and collagenous tissue may substantially completely overgrow the tissue-ingrowth region, growing over and affixing the tissue-ingrowth region to the tissue. In one implementation, the tissue-ingrowth region comprises a plurality of alveoli or apertures visible to the naked eye, through or over which the host tissue can grow and achieve substantial fixation.

As an example, pores may be formed into the tissue-ingrowth region by punching or otherwise machining, or by using laser energy. Non-smooth surfaces may be formed, for example, by abrading the tissue-ingrowth region with a relatively course surface (e.g., having a 40 or, preferably, higher grit sandpaper-like surface) or, alternatively, non-smooth surfaces may be generated by bringing the tissue-ingrowth region up to its softening or melting temperature and imprinting it with a template (to use the same example, a sandpaper-like surface). The imprinting may occur, for example, during an initial formation process or at a subsequent time.

On the other hand, the adhesion-resistant region can be formed to have a closed, continuous, smooth and/or non-porous surface. In an illustrative embodiment, at least a portion of the adhesion-resistant region is smooth comprising no protuberances, alveoli or vessel-permeable pores, so as to attenuate occurrences of adhesions between the tissue-ingrowth region and host tissues.

In a molding embodiment, one side of the press may be formed to generate any of the tissue-ingrowth region surfaces discussed above and the other side of the press may be formed to generate an adhesion-resistant region surface as discussed above. Additional features (e.g., roughening or forming apertures) may subsequently be added to further define the surface of, for example, the tissue-ingrowth region. In an extrusion embodiment, one side of the output orifice may be formed (e.g. ribbed) to generate a tissue-ingrowth region (wherein subsequent processing can further define the surface such as by adding transverse ribs/features and/or alveoli) and the other side of the orifice may be formed to generate an adhesion-resistant biodegradation region surface. In one embodiment, the adhesion-resistant region is extruded to have a smooth surface and in another embodiment the adhesion-resistant region is further processed (e.g., smoothed) after being extruded.

### B. Surface Composition (Function):

As presently embodied, the tissue-ingrowth region comprises a first material, and the adhesion-resistant region comprises a second material which is different from the first material. In modified embodiments, the tissue-ingrowth region and the adhesion-resistant region may comprise the same or substantially the same materials. In other embodiments, the tissue-ingrowth region and the adhesion-resistant region may comprise different materials resulting from, for example, an additive having been introduced to at least one of the tissue-ingrowth region and the adhesion-resistant region.

According to an implementation of the present invention, the adhesion-resistant region is constructed to minimize an occurrence of adhesions of host tissues (e.g., internal body viscera) to the surgical prosthesis. In modified embodiments, the adhesion-resistant region and the tissue-ingrowth region of the surgical prosthesis may be formed of the same material or relatively less divergent materials, functionally speaking, and the adhesion-resistant region may be used in conjunction with an anti-inflammatory gel agent applied, for example, onto the adhesion-resistant region at a time of implantation of the surgical prosthesis. According to other broad embodiments, the adhesion-resistant region and the tissue-ingrowth region may be formed of any materials or combinations of materials disclosed herein (including embodiments wherein the two regions share the same layer of material) or their substantial equivalents, and the adhesion-resistant region may be used in conjunction with an anti-inflammatory gel agent applied, for example, onto the adhesion-resistant region at a time of implantation of the surgical prosthesis.

The tissue-ingrowth region can be formed of similar and/or different materials to those set forth above, to facilitate strength, longevity or lack thereof, and/or direct post-surgical cell colonization via, for example, invoking a substantial fibroblastic reaction in the host tissue. In an illustrated embodiment, the tissue-ingrowth region is constructed to be substantially incorporated into the host tissue and/or to substantially increase the structural integrity of the surgical prosthesis. Following implantation of the surgical prosthesis, body tissues (e.g., subcutaneous tissue and/or the exterior fascia) commence to incorporate themselves into the tissue-ingrowth region. While not wishing to be limited by theory, it is believed that the body, upon sensing the presence of the tissue-ingrowth region of the present invention, is disposed to send out fibrous tissue which grows in, around and/or through and at least partially entwines itself with the tissue-ingrowth region. In this manner, the surgical prosthesis can become securely attached to the host body tissue.

Regarding different materials, according to an aspect of the present invention, the tissue-ingrowth region can comprises a biodegradable (e.g., resorbable) polymer composition having one or more different characteristics than that or those of a biodegradable (e.g., resorbable) polymer composition of the adhesion-resistant region. The different characteristics may include (1a) time or rate of biodegradation affected by additives, (1b) time or rate of biodegradation affected by polymer structures/compositions, (2) polymer composition affecting strength or structural integrity, and (3) ability to facilitate fibroblastic reaction.

In accordance with a method of the present invention, the surgical prosthesis can be used to facilitate repair of, for example, a hernia in the ventral region of a body. An implanted surgical prosthesis having both an adhesion-resistant region disposed on one side and having a tissue-ingrowth region disposed on a second side of the surgical prosthesis can be provided. The abdominal wall can include muscle enclosed and held in place by an exterior fascia and an interior fascia. An interior layer, called the peritoneum, can cover the interior side of the interior fascia. The peritoneum is a softer, more pliable layer of tissue that forms a sack-like enclosure for the intestines and other internal viscera. A layer of skin and a layer of subcutaneous fat cover the exterior fascia.

Surgical repair of a soft tissue defect (e.g., a hernia) can be performed by using, for example, conventional techniques or advanced laparoscopic methods to close substantially all of a soft tissue defect. According to one implementation, an incision can be made through the skin and subcutaneous fat, after which the skin and fat can be peeled back followed by any protruding internal viscera (not shown) being positioned internal to the hernia. In certain implementations, an incision can be made in the peritoneum followed by insertion of the surgical prosthesis into the hernia opening so that the surgical prosthesis is centrally located in the hernia opening. One or both the tissue-ingrowth region and the adhesion-resistant region may be attached by, e.g., suturing to the same layer of the abdominal wall, e.g., the relatively-strong exterior fascia. Alternatively, the adhesion-resistant region may be attached to another member, such as the interior fascia and/or the peritoneum. The tissue-ingrowth region can be surgically attached to the exterior fascia while the adhesion-resistant region can be attached to the tissue-ingrowth region and/or optionally to the exterior fascia using, e.g., heat bonding, suturing, and/or other affixation protocols disclosed herein or their substantial equivalents. Those possessing skill in the art will recognize that other methods of sizing/modifying/orientating/attaching a surgical prosthesis of this invention may be implemented according to the context of the particular surgical procedure.

The size of the surgical prosthesis typically will be determined by the size of the defect. Use of the surgical prosthesis in a tension-free closure may be associated with less pain and less incidence of post surgical fluid accumulation. Exemplary sutures may be implemented to at least partially secure the surgical prosthesis to the abdominal wall structure. The sutures can be implemented so that no lateral tension is exerted on the exterior fascia and/or muscle. When disrupted, the skin and fat may be returned to their normal positions, with, for example, the incisional edges of the skin and fat being secured to one another using suitable means such as subsurface sutures.

In modified embodiments of the present invention, one or both of the tissue-ingrowth region and the adhesion-resistant region of the surgical prosthesis, can be heat bonded (or in a modified embodiment, otherwise attached, such as by suturing). Heat bonding may be achieved, for example, with a bipolar electro-cautery device, ultrasonicly welding, or similar sealing between the tissue-ingrowth region and the adhesion-resistant region and/or directly to surrounding tissues. Such a device can be used to heat the surgical prosthesis at various locations, such as at edges and/or at points in the middle, at least above its glass transition temperature, and preferably above its softening point temperature. The material is heated, e.g., along with adjacent tissue, such that the two components bond together at their interface. The heat bonding may also be used initially, for example, to secure the tissue-ingrowth region to the adhesion-resistant region. Since the tissue-ingrowth region serves more of a load-bearing function, a few typical embodiments may exclude heat-bonding as the sole means for securing this region to host tissues. In other embodiments, the technique of heat bonding the surgical prosthesis to itself or body tissue may be combined with another attachment method for enhanced anchoring. For example, the surgical prosthesis may be temporarily affixed in position using two or more points of heat bonding using an electro-cautery device, and sutures, staples or glue can subsequently (or in other embodiments, alternatively) be added to secure the surgical prosthesis into place.

The tissue-ingrowth region and the adhesion-resistant region may be arranged to form more than one layer or substantially one layer, or the regions may both belong to a single, integrally formed layer. For example, the tissue-ingrowth region and the opposing adhesion-resistant region may be arranged in two layers, wherein one of the regions is disposed on top of, and opposite to, the other region.

In one embodiment, the tissue-ingrowth region and the adhesion-resistant region may be combined on a single side of the surgical prosthesis in, for example, substantially one layer, wherein the regions are adjacent each other on one side of the surgical prosthesis. As a slight deviation, a surgical prosthesis having a tissue-ingrowth region on at least one (and preferably, both) side(s) thereof may be manufactured using any of the techniques described herein and, subsequently, an adhesion-resistant region may be formed on, e.g., one side, by smoothing, filling, or otherwise processing an area of the tissue-ingrowth region with a suitable material as disclosed herein or technique (e.g., coating or filling with a liquid or flowable polymer composition, and/or mechanically smoothing) to thereby form an adhesion-resistant region having adhesion-resistant properties relative to those of the tissue-ingrowth region.

Similarly, a patch of adhesion-resistant region may be sized and affixed (e.g., heat bonded, such as with a bipolar electro-cautery device, ultrasonicly welded, or similarly affixed) at a time of implantation directly to at least one of the tissue-ingrowth region and surrounding host tissues. In modified embodiments, the affixing may be accomplished using, for example, press or adhesive bonding, or sutures. In further embodiments, at least part of the affixing may occur at a time of manufacture of the surgical prosthesis before packaging. The patch of adhesion-resistant region alternatively may be partially affixed (e.g., using techniques enumerated in this paragraph) at, for example, a non-perimeter or central area thereof to an area (e.g., a non-perimeter or central area) of the tissue-ingrowth region, so that a surgeon can trim the adhesion-resistant region (and/or the tissue-ingrowth region) at a time of implantation while the adhesion-resistant biodegradable implant is affixed to the tissue-ingrowth region. For instance, a tissue-ingrowth region may substantially surround an adhesion-resistant region on one side of the surgical prosthesis, and only a tissue-ingrowth region may be formed on the other side of the surgical prosthesis. In such an implementation, the adhesion-resistant region of the surgical prosthesis can be sized and shaped so as to substantially cover any opening created by the soft tissue defect, with the tissue-ingrowth regions facilitating surgical attachment to, and incorporation into, the host tissue on at least one side of, and, preferably, on both sides of, the surgical prosthesis.

In modified embodiments, the tissue-ingrowth region and/or the adhesion-resistant region on a given surface or surfaces of the surgical prosthesis each may be of any size or shape suited to fit the particular soft tissue defect. For example, either of the tissue-ingrowth region and/or the adhesion-resistant region on a given surface of the surgical prosthesis may have shapes of ovals, rectangles and various complex or other shapes wherein, for each such implementation, the two regions may have essentially the same, or different, proportions and/or dimensions relative to one another.

In general, various techniques may be employed to produce the surgical prosthesis, which typically has one or two layers defining the tissue-ingrowth region and the adhesion-resistant region. Useful techniques include solvent evaporation methods, phase separation methods, interfacial methods, extrusion methods, molding methods, injection molding methods, heat press methods and the like as known to those skilled in the art. The tissue-ingrowth region and the adhesion-resistant region may comprise two distinct layers or may be integrally formed together as one layer.

The tissue-ingrowth region and the adhesion-resistant region may be partially or substantially entirely formed or joined together. Joining can be achieved by mechanical methods, such as by suturing or by the use of metal clips, for example, hemoclips, or by other methods, such as chemical or heat bonding.

The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. As iterated above, any feature or combination of features described and referenced herein are included within the scope of the present invention provided that the features included in any such combination are not mutually inconsistent as will be apparent from the context, this specification, and the knowledge of one of ordinary skill in the art. For example, any of the implants and implant components, sub-components, or uses, and any particulars or features thereof, or other features, including method steps and techniques, may be used with any other structure and process described or referenced herein, in whole or in part, in any combination or permutation. Accordingly, the present invention is not intended to be limited by the disclosed embodiments, but is to be defined by reference to the appended claims.

What is claimed is:

1. A resorbable scar-tissue reduction micro-membrane system for attenuating or preventing a formation of post-surgical scar tissue between a healing post-surgical site and adjacent surrounding tissue following an in vivo surgical procedure on the post-surgical site, the system having a pre-implant configuration, which is defined as a configuration of the system immediately before the system is formed between the post-surgical site and the adjacent surrounding tissue, the system comprising a substantially planar membrane of resorbable polymer base material having a first substantially-smooth side and a second substantially-smooth side, the substantially planar membrane of resorbable polymer base material comprising a single layer of resorbable polymer base material between the first substantially-smooth side and the second substantially-smooth side, the single layer of resorbable polymer base material including (a) at least one hydrophobic block with one or more of a lactide and a glycolide and (b) at least one hydrophilic blocks with a polyethylene glycol, and further including a form of one or more of a triblock copolymer and a starblock copolymer.

2. The resorbable scar-tissue reduction micro-membrane system as set forth in claim 1, wherein:
the single layer of resorbable polymer base material has a substantially uniform composition;
a thickness of the single layer of resorbable polymer base material, measured between the first substantially-smooth side and the second substantially-smooth side, is between about 10 microns and about 300 microns;
the single layer of resorbable polymer base material is non-porous; and
the single layer of resorbable polymer base material is adapted to maintain a smooth-surfaced barrier between the healing post-surgical site and the adjacent surrounding tissue for a relatively extended period of time sufficient to attenuate or eliminate any formation of scar tissue between the post-surgical site and the adjacent surrounding tissue.

3. A resorbable scar-tissue reduction membrane system for attenuating or preventing a formation of post-surgical scar tissue between a healing post-surgical site and adjacent surrounding tissue following an in vivo surgical procedure on the post-surgical site, the system having a pre-implant configuration, which is defined as a configuration of the system immediately before the system is formed between the post-surgical site and the adjacent surrounding tissue, the system comprising a substantially planar membrane of resorbable polymer base material having a first substantially-smooth side and a second substantially-smooth side, the substantially planar membrane of resorbable polymer base material comprising a layer of resorbable polymer base material between the first substantially-smooth side and the second substantially-smooth side, the single layer of resorbable polymer base material including (a) at least one hydrophobic block with one or more of a lactide, a glycolide, or a mixture of a lactide and a glycolide and (b) at least one hydrophilic blocks with a polyethylene glycol, and further including a form of a 4plus block copolymer.

4. The resorbable scar-tissue reduction micro-membrane system as set forth in claim 3, wherein:
the single layer of resorbable polymer base material has a substantially uniform composition;
a thickness of the single layer of resorbable polymer base material, measured between the first substantially-smooth side and the second substantially-smooth side, is between about 10 microns and about 300 microns;
the single layer of resorbable polymer base material is non-porous; and
the substantially planar membrane of resorbable polymer base material is disposed in a package.

5. The resorbable scar-tissue reduction micro-membrane system as set forth in claim 3, wherein the single layer of resorbable polymer base material comprises (i) a first hydrophobic block with one or more of a lactide, a glycolide, or a mixture of a lactide and a glycolide and (ii) a plurality of second hydrophilic blocks with polyethylene glycols.

6. The resorbable scar-tissue reduction micro-membrane system as set forth in claim 5, wherein the single layer of resorbable polymer base material comprises a starblock copolymer.

7. The resorbable scar-tissue reduction micro-membrane system as set forth in claim 3, wherein the single layer of resorbable polymer base material comprises a starblock copolymer having (i) a first hydrophobic PLA/PGA block and (ii) three or more second hydrophilic PEG blocks.

8. The resorbable scar-tissue reduction micro-membrane system as set forth in claim 3, wherein the single layer of resorbable polymer base material comprises (i) a first hydrophobic block with at least one polyethylene glycol and (ii) a plurality of second hydrophilic blocks each with one or more of a lactide, a glycolide, or a mixture of a lactide and a glycolide.

9. The resorbable scar-tissue reduction micro-membrane system as set forth in claim 8, wherein the single layer of resorbable polymer base material comprises a starblock copolymer.

10. The resorbable scar-tissue reduction micro-membrane system as set forth claim 3, wherein the single layer of resorbable polymer base material comprises a starblock copolymer having (i) at least a first hydrophobic PEG block and (ii) three or more second hydrophilic PLA/PGA blocks.

11. The resorbable scar-tissue reduction micro-membrane system as set forth in claim 3, wherein the single layer of resorbable polymer base material includes a triblock or a 4plus block copolymer comprising a first hydrophobic block of one or more of a lactide, a glycolide, or a mixture of a lactide and a glycolide, a second hydrophilic block of at least one polyethylene glycol, and a third hydrophobic block of one or more of a lactide, a glycolide, a mixture of a lactide and a glycolide, and a polyethylene glycol.

12. The resorbable scar-tissue reduction micro-membrane as set forth in claim 3, wherein the maximum thickness is about 100 microns.

13. The resorbable scar-tissue reduction micro-membrane as set forth in claim 3, wherein the maximum thickness is about 200 microns.

14. The resorbable scar-tissue reduction micro-membrane as set forth in claim 3, wherein the single layer of resorbable polymer base material is not fluid permeable.

15. The resorbable scar-tissue reduction micro-membrane as set forth in claim 3, wherein the single layer of resorbable polymer base material comprises at least one of a chemotactic substance for influencing cell-migration, an inhibitory substance for influencing cell-migration, a mitogenic growth factor for influencing cell proliferation, a growth factor for influencing cell differentiation, and factors which promote neoangiogenesis.

16. The resorbable scar-tissue reduction micro-membrane system as set forth in claim 3, wherein the resorbable scar-tissue reduction micro-membrane system is sealed in a sterile packaging.

17. The resorbable scar-tissue reduction micro-membrane system as set forth in claim 3, wherein the single layer of resorbable polymer base material comprises a plurality of holes disposed along an edge of the single layer of resorbable polymer base material.

18. The resorbable scar-tissue reduction micro-membrane system as set forth in claim 3, wherein the single layer of resorbable polymer base material does not comprise any holes substantially away from an edge of the single layer of resorbable polymer base material.

19. The resorbable scar-tissue reduction micro-membrane system as set forth in claim 3, wherein the edge extends around the single layer of resorbable polymer base material.

20. The resorbable scar-tissue reduction micro-membrane system as set forth in claim 3, wherein a slit is formed in a periphery of the single layer of resorbable polymer base material so that the edge extends along the slit.

21. The resorbable scar-tissue reduction micro-membrane system as set forth in claim 3, wherein:
the single layer of resorbable polymer base material further comprises a plurality of holes disposed away from the edge;
each of the holes near the periphery has a first diameter;
each of the holes near the center has a second diameter; and
the first diameters are greater than the second diameters.

22. The resorbable scar-tissue reduction micro-membrane system as set forth in claim 3, wherein a slit is formed in a periphery of the single layer of resorbable polymer base material so that the edge extends along the slit.

23. The resorbable scar-tissue reduction micro-membrane system as set forth in claim 3, wherein the single layer of resorbable polymer base material comprises a slit disposed in the non-porous base material.

24. The resorbable scar-tissue reduction micro-membrane system as set forth in claim 3, wherein the single layer of resorbable polymer base material is cut to have a size and shape suitable for snugly and anatomically fitting over an anatomic structure to thereby attenuate or prevent formation of scar tissue between the anatomic structure and surrounding tissue, and is sealed in a sterile packaging.

25. The resorbable scar-tissue reduction micro-membrane system as set forth in claim 3, wherein the single layer of resorbable polymer base material is cut with tabs to be folded over and around an anatomic structure.

26. The resorbable scar-tissue reduction micro-membrane system as set forth in claim 3, wherein the single layer of resorbable polymer base material comprises at least one notch disposed in the non-porous base material.

27. The resorbable scar-tissue reduction micro-membrane system as set forth in claim 3, wherein the single layer of resorbable polymer base material comprises a plurality of notches disposed in the non-porous base material.

28. The resorbable scar-tissue reduction micro-membrane system as set forth in claim 3, wherein the single layer of resorbable polymer base material is cut to have a non-rectangular and non-circular shape and is sealed in a sterile packaging.

29. The resorbable scar-tissue reduction micro-membrane system as set forth in claim 3, wherein the resorbable scar-tissue reduction micro-membrane system further includes another membrane, which comprises a maximum thickness less than 2000 microns and which is permeable.

30. The resorbable scar-tissue reduction micro-membrane system as set forth in claim 3, wherein the other membrane is a bridging membrane.

31. The resorbable scar-tissue reduction micro-membrane system as set forth in claim 3, wherein the other membrane is fluid permeable.

32. The resorbable scar-tissue reduction micro-membrane system as set forth in claim 3, wherein the other membrane is cell permeable.

33. The resorbable scar-tissue reduction micro-membrane system as set forth in claim 3, wherein the other membrane is vessel permeable.

34. The resorbable scar-tissue reduction micro-membrane system as set forth in claim 3, wherein the other membrane comprises a thickness between 500 microns and 2000 microns.
